(19) United States
(12) Patent Application Publication
(10) Pub. No.: US 2026/0360165 A1
(43) Pub. Date: Nov. 12, 2026

(54) FLOW CELL FOR REAL-TIME MONITORING OF MICROSCOPIC LARVAE IN TAP WATER AND AQUA SMART MACHINE VISION SYSTEM INCLUDING THE SAME

(71) Applicant: MSTEC Co., Ltd., Uiwang-si (KR)

(72) Inventors: Min Soo LEE, Hwaseong-si (KR); Gwang Seok KIM, Namyangju-si (KR); Su Eung RIM, Yongin-si (KR); Na Kyoung LEE, Hwaseong-si (KR); Na Yeon LEE, Hwaseong-si (KR)

(73) Assignee: MSTEC Co., Ltd., Uiwang-si (KR)

(21) Appl. No.: 19/669,924

(22) Filed: May 6, 2026

(30) Foreign Application Priority Data
May 9, 2025 (KR) 10-2025-0060280
Apr. 7, 2026 (KR) 10-2026-0062862

(51) Int. Cl. G06V 20/69; G01N 15/1429; G01N 15/1434; G01N 33/18; G06V 10/26; G06V 10/70; H04N 23/56; G01N 15/10

(52) U.S. Cl. CPC G06V 20/698; G01N 15/1429; G01N 15/1436; G01N 33/18; G06V 10/26; G06V 10/70; G06V 20/693; G06V 20/695; H04N 23/56; G01N 2015/1006

(57) ABSTRACT

A flow cell with an integrated inlet-side water distribution block for real-time monitoring of microscopic larvae in tap water features water passage holes spaced progressively more closely from the center toward both ends, thereby uniformly distributing large volumes of tap water across the full width without requiring a separate external distributor. In a preferred embodiment, an outlet-side water distribution block is also provided to maintain uniform flow. A machine vision system incorporating the flow cell captures real-time images of flowing tap water, detects target objects, and classifies them as larval organisms or non-larval foreign matter using a CNN-based artificial intelligence object detection algorithm that assigns confidence scores. The system further includes a cloud computing web server, a web-based visualization platform, and a real-time notification platform.

### STATEMENT REGARDING GOVERNMENT SUPPORT

This invention was supported by the Korea Technology and Information Promotion Agency for SMEs funded by the Ministry of SMEs and Startups of the Republic of Korea [Research Project: “SMEs Technology Innovation Development Project (Market Response Type)”; Research Subject: “Development of Smart Machine Vision System for Real-Time Monitoring of Tap Water Larvae”; Project Serial Number: 242000666; Research Subject Number: RS-2024-00508739].

### CROSS-REFERENCE TO RELATED APPLICATIONS

This application claims priority under 35 U.S.C. § 119(a) to the following Korean Patent Applications, the disclosures of which are incorporated herein by reference in their entireties:
(1) Korean Patent Application No. 10-2025-0060280, filed May 9, 2025; and
(2) Korean Patent Application No. 10-2026-0062862, filed Apr. 7, 2026.

### FIELD OF THE INVENTION

The present invention relates to a flow cell with an integrated water distribution block for real-time monitoring of microscopic larvae in tap water, and to a machine vision system including the same. More particularly, the invention relates to a flow cell that enables real-time detection of foreign matter in tap water using a vision camera while the tap water flows through a transparent channel, and to a machine vision system that automatically detects microscopic aquatic larvae such as chironomid midge larvae in tap water and notifies users or administrators.

### BACKGROUND OF THE INVENTION

Although tap water is supplied to users after purification at water treatment plants, microscopic larvae of chironomid midges and other aquatic insects (hereinafter referred to as “larva” or “larval organisms”) may infiltrate or proliferate during the purification process or during distribution, potentially contaminating the tap water supply. Accordingly, there is a growing need for real-time monitoring of tap water to detect such larval organisms.

Various technologies have been developed for detecting larval organisms by monitoring foreign matter in tap water. One prior art approach is disclosed in Korean Patent Publication No. 2022-0072654, entitled “Artificial Intelligence-Based Larva Monitoring System and Method” (hereinafter “Patent Document 1”).

Patent Document 1 discloses a system comprising: one or more cameras for capturing images of the interior of a water storage tank; a video conversion unit for converting captured image data into motion video; a biological determination unit for comparing the motion video against pre-stored training data to determine whether aquatic organisms are present; and a transmission unit for forwarding organism information to a monitoring unit when organisms are detected.

A shortcoming of Patent Document 1 is that it relies solely on movement in image data to detect larval organisms. Because microscopic foreign particles suspended in water also move with the water current, such movement can be misidentified as larval activity. Repeated false detections undermine the reliability of the larva detection system.

To address these shortcomings, the present applicant previously obtained Korean Patent Registration No. 10-2531861, entitled “Real-Time Monitoring System for Aquatic Larvae” (hereinafter “Patent Document 2”).

Patent Document 2 discloses a system comprising: an image capturing unit that photographs tap water flowing through a transparent tube using multiple cameras; a data transmission unit; a data storage unit; a visualization unit that renders target objects as images from two or more directions; a data analysis unit that classifies target objects as larval organisms or non-larval foreign matter and identifies larva type through machine learning and deep learning comparison against a larva database; and a result notification unit.

While Patent Document 2 improves detection accuracy by rendering target objects stereoscopically, it encounters difficulty detecting larval organisms quickly when large volumes of tap water are supplied at high flow rates.

Furthermore, Patent Document 2 requires a separate water distributor upstream of the transparent tube to spread tap water uniformly across the tube’s width. If foreign matter accumulates or air pockets form inside the distributor’s channels, the channels can become partially blocked, disrupting uniform supply to the transparent tube.

Accordingly, there remains a need for a flow cell with an integrated water distribution block capable of uniformly distributing large volumes of tap water without a separate external distributor, and for a machine vision system capable of quickly and accurately detecting larval organisms in tap water flowing through such a flow cell.

### SUMMARY OF THE INVENTION

In one aspect, the present invention provides a flow cell with an integrated water distribution block for real-time monitoring of microscopic larvae in tap water. The flow cell comprises a pair of main body frames, a pair of transparent glass plates, a tap water inlet unit, a tap water outlet unit, side sealing blocks, and an inlet-side water distribution block. The water passage holes of the inlet-side distribution block are spaced progressively more closely from the center toward both ends, thereby uniformly distributing tap water across the full width of the flow cell without a separate external distributor.

In a preferred embodiment, the outlet unit further includes an outlet-side water distribution block to achieve uniform flow at both the inlet and outlet ends.

In another aspect, the present invention provides a machine vision system for real-time monitoring of microscopic larvae in tap water, comprising a machine vision inspection device equipped with the flow cell, a cloud computing web server, an analysis data visualization web platform, and a notification platform.

### BRIEF DESCRIPTION OF THE DRAWINGS

FIG. 1 is a block diagram illustrating an embodiment of the machine vision system for real-time monitoring of microscopic larvae in tap water according to the present invention.

FIG. 2 is a block diagram illustrating an embodiment of the machine vision inspection device according to the present invention.

FIGS. 3 and 4 are diagrams illustrating a first embodiment of the flow cell according to the present invention.

FIG. 5 is a diagram illustrating larva detection and classification through the AI object detection algorithm according to the present invention.

FIGS. 6 and 7 are cross-sectional views illustrating a second embodiment of the flow cell according to the present invention.

FIG. 8 is a plan view of the second embodiment of the flow cell according to the present invention.

FIG. 9 is a diagram illustrating the tap water inlet unit and the inlet-side water distribution block of the second embodiment of the flow cell according to the present invention.

FIG. 10 is a diagram illustrating a first configuration of the tap water outlet unit of the second embodiment of the flow cell according to the present invention.

FIG. 11 is a diagram illustrating a second configuration of the tap water outlet unit further including an outlet-side water distribution block according to the present invention.

FIG. 12 is a diagram illustrating the second embodiment of the flow cell with both an inlet-side and outlet-side water distribution block, representing the preferred embodiment according to the present invention.

### DETAILED DESCRIPTION OF THE INVENTION

The following description sets forth exemplary embodiments of the present invention with reference to the accompanying drawings. Like reference numerals designate like or corresponding elements throughout the drawings.

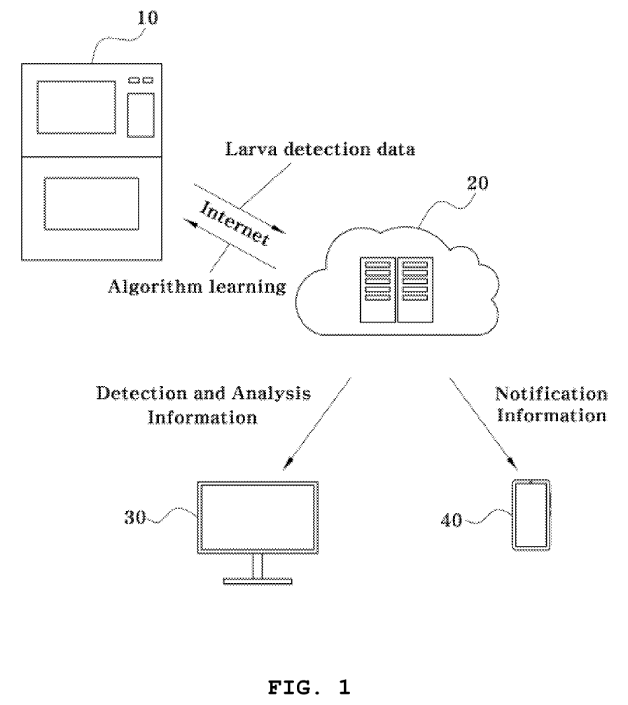

As shown in FIG. 1, the machine vision system according to the present invention comprises a machine vision inspection device (10), a cloud computing web server (20), an analysis data visualization web platform (30), and a notification platform (40). The machine vision inspection device (10) is installed at a tap water supply facility, such as a residential water supply line or a water treatment plant, and captures real-time images of tap water flowing through the supply pipe for automatic analysis of larval organisms and foreign matter using artificial intelligence.

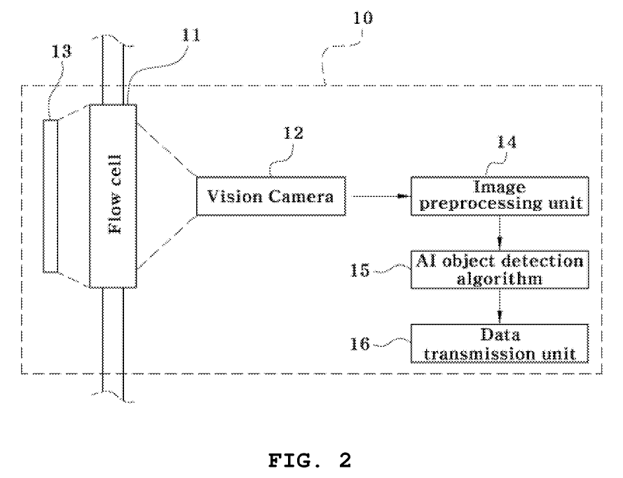

As shown in FIG. 2, the machine vision inspection device (10) comprises: a housing with an internal accommodation space; a flow cell (11) installed inside the housing and connected to the supply pipe so that tap water passes therethrough; one or more vision cameras (12) positioned on one side of the flow cell (11) to capture real-time images of the flowing tap water; a lighting device (13) positioned on the opposite side of the flow cell (11) to provide transmitted illumination so that the camera(s) can clearly image foreign matter in the tap water; an image preprocessing unit (14) inside the housing that removes noise from captured images and isolates images of target objects; an AI object detection algorithm (15) inside the housing that automatically classifies each target object as a larva or non-larval foreign matter; and a data transmission unit (16) inside the housing that transmits larva detection data to the cloud computing web server (20) via wired or wireless internet.

In one embodiment, the housing is divided into upper and lower compartments. The flow cell (11) and vision camera(s) (12) are housed in the lower compartment, which is accessed through a door (not shown) on one side. An inspection window (not shown) in the door permits visual checking of the flow cell (11) and camera(s) (12) without opening the door. The upper compartment houses the hardware for the image preprocessing unit (14) and AI object detection algorithm (15). A display (not shown) on one face of the upper compartment outputs real-time camera images, detected larva images, and system status information. A control panel (not shown) for operating the device is provided adjacent to the display.

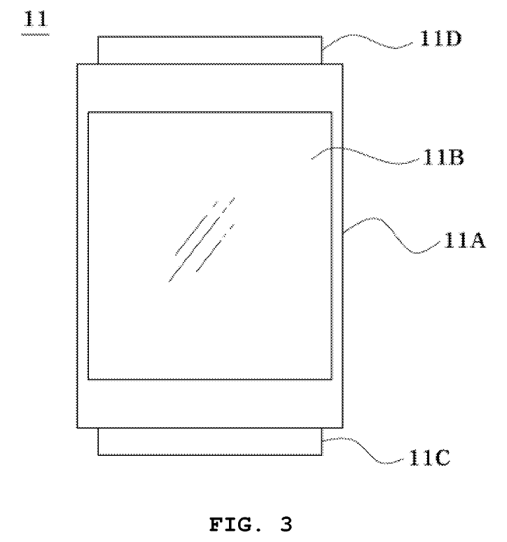

As shown in FIGS. 3 and 4, a first embodiment of the flow cell (11) comprises: a pair of main body frames (11A) each formed in a rectangular plate shape having a through-hole in its center, installed spaced apart in the front-rear direction; a pair of transparent glass plates (11B) covering the respective through-holes of the frames (11A); a tap water inlet unit (11C) on the lower side of the frames (11A) to supply tap water between the glass plates (11B); and a tap water outlet unit (11D) on the upper side to discharge the tap water. The lighting device (13) faces the side of the frames (11A) opposite the vision camera(s) (12), enabling clear transmitted-light imaging of the flowing tap water. The inlet unit (11C) and outlet unit (11D) each include a plurality of inlet pipes (11C’) and outlet pipes (11D’), respectively, through which large volumes of tap water are naturally distributed across the interior of the frames (11A).

The image preprocessing unit (14) applies a noise filter to real-time images received from the vision camera(s) (12) and may additionally upscale image resolution to facilitate identification of target objects.

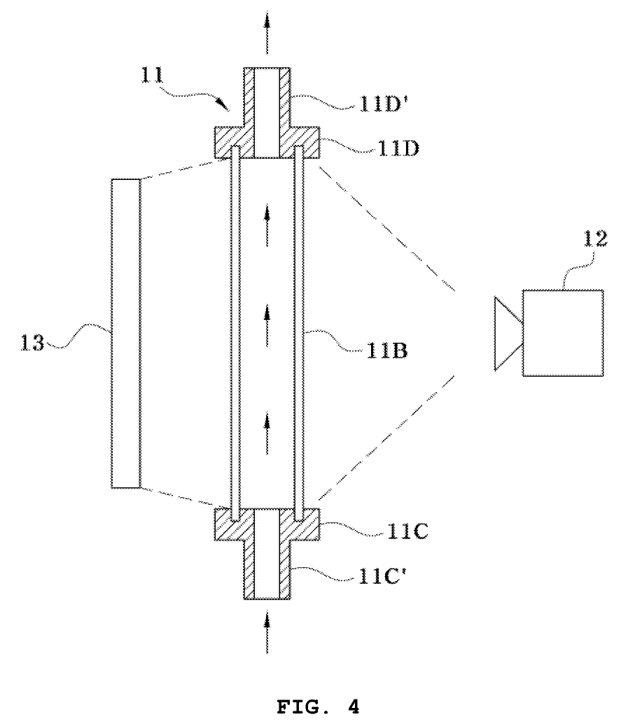

As shown in FIG. 5, the AI object detection algorithm (15) divides each preprocessed image into a grid of S×S square areas and determines whether a target object is present in each area. When a target object is detected, a snap image of that area is captured and submitted to the algorithm (15), which assigns a confidence score to each target object based on comparison with trained larva models, and classifies the object as a larva or non-larval foreign matter and, if a larva, identifies its species. The algorithm (15) employs a Convolutional Neural Network (CNN)-based deep learning model trained on a dataset comprising thousands of labeled images of various larva species, body orientations, sizes, and lighting conditions, as well as non-larval foreign matter.

A target object whose confidence score meets or exceeds a predefined classification threshold is classified as a larva; one whose score falls below the threshold is classified as non-larval foreign matter. When the same confidence score recurs for a set number of consecutive frames, classification is suspended and the data is forwarded to the cloud computing web server (20) for final determination by a user or administrator through the web platform (30) and notification platform (40).

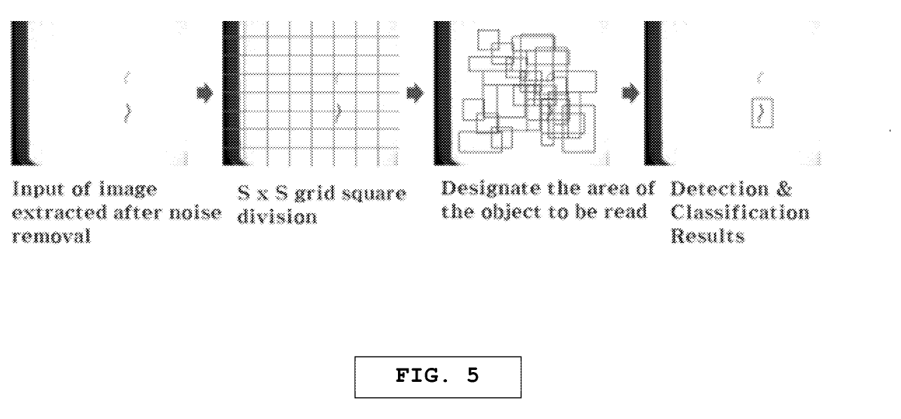

A second, and preferred, embodiment of the flow cell (110) is shown in FIGS. 6 and 7. The second embodiment improves upon the first by incorporating an inlet-side water distribution block (111F) for uniform flow distribution without a separate external distributor. The flow cell (110) comprises: a pair of main body frames (111A) each formed in a rectangular plate shape having a through-hole in its center, installed facing each other and spaced apart in the front-rear direction; a pair of transparent glass plates (111B) covering the respective through-holes; a tap water inlet unit (111C) on the lower side of the frames (111A); a tap water outlet unit (111D) on the upper side; side sealing blocks (111E) installed along the full height on both lateral sides between the frames (111A); and the inlet-side water distribution block (111F) installed horizontally inside the inlet unit (111C) or between the frames (111A), having a plurality of water passage holes to distribute tap water uniformly across the width of the flow cell.

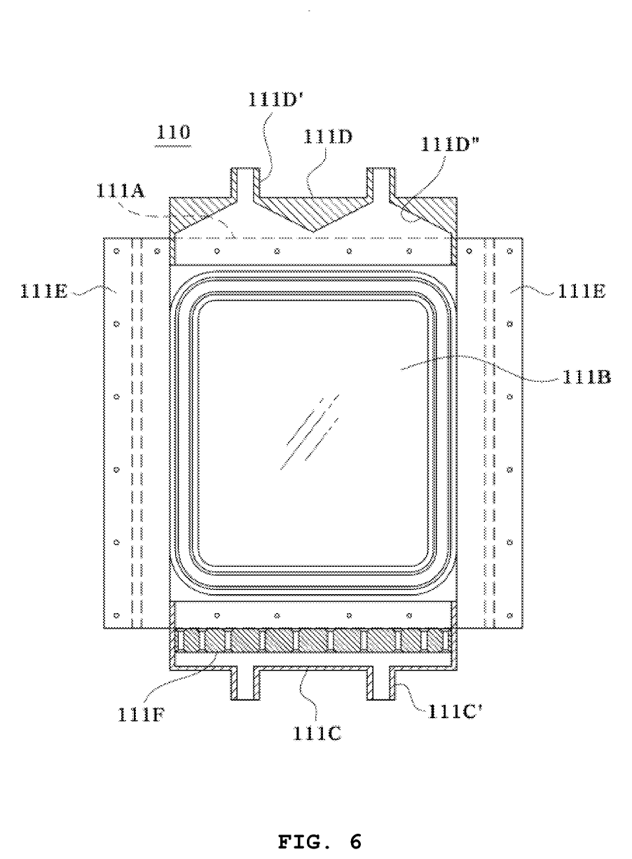

As shown in FIG. 8, sealing members (not shown) are disposed along the perimeter of the through-holes where the frames (111A) contact the glass plates (111B), and along both lateral surfaces where the frames (111A) contact the side sealing blocks (111E). Fasteners (not shown) passing through both frames (111A) clamp the assembly together, ensuring an airtight interior. Alternatively, the side sealing blocks (111E) may be formed of resilient rubber, eliminating the need for separate sealing members.

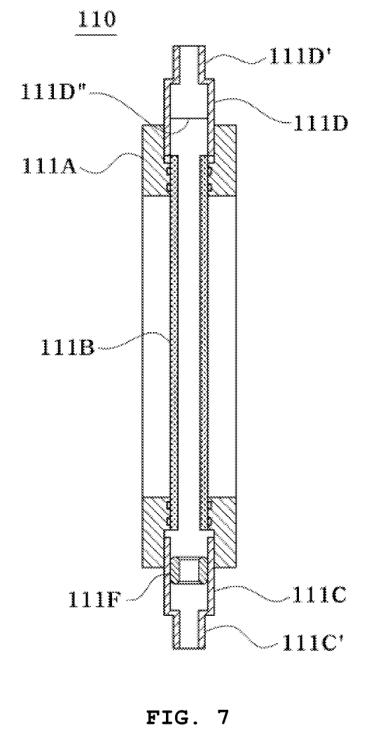

As shown in FIG. 9, the tap water inlet unit (111C) includes a pair of inlet pipes (111C’). The inlet-side water distribution block (111F) is spaced from the inner surface of the inlet unit (111C) by a gap (G) and has a plurality of oblong water passage holes oriented vertically along its length. The passage holes are positioned offset from the inlet pipes (111C’), so that water issuing from the pipes cannot pass directly through the holes without first spreading laterally along the face of the block (111F).

The passage holes are spaced progressively more closely from the center toward both ends of the block (111F)—end spacing (H2) being narrower than center spacing (H1)—so that more holes are present where water pressure is lower, at positions farther from the inlet pipes (111C’). The combined effect of the pressure gradient and hole density gradient yields a uniform volumetric flow rate through the holes across the full width of the flow cell.

The gap (G) between the block (111F) and the inner surface of the inlet unit (111C) is adjustable to accommodate different supply flow rates. In one embodiment, the gap (G) is set by threaded bolts or adjustment screws that bear against the inner surface of the inlet unit (111C), enabling the operator to tune the pre-distribution volume without disassembling the flow cell.

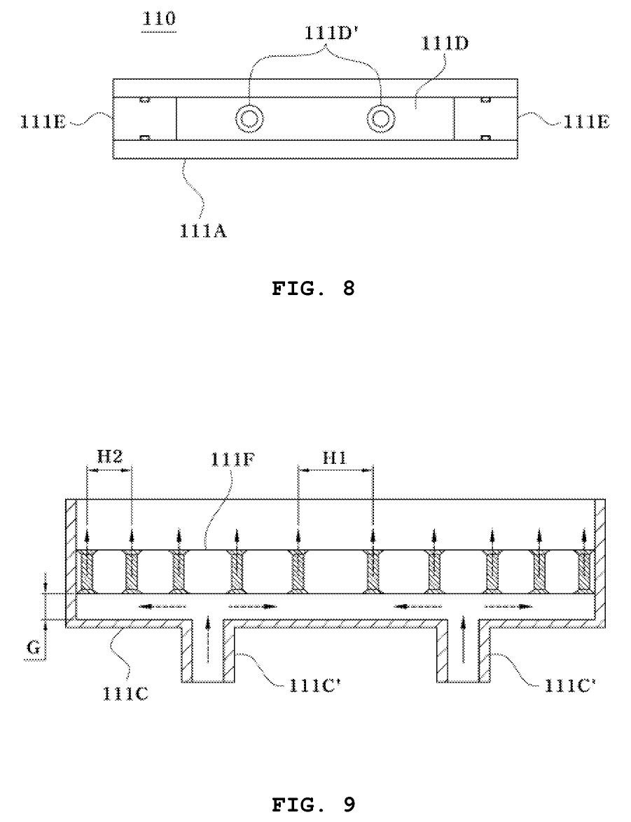

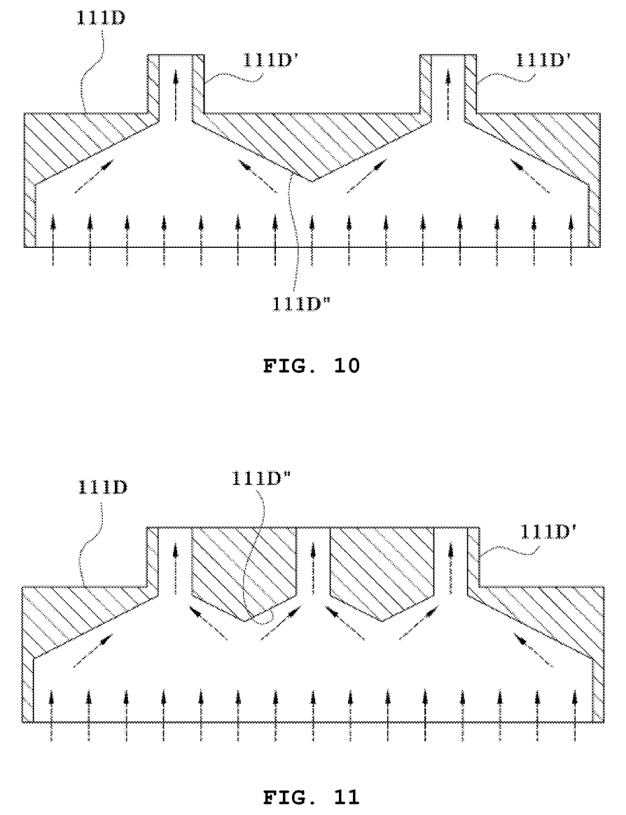

As shown in FIG. 10, the tap water outlet unit (111D) includes a pair of drain pipes (111D’) with drainage holes. Discharge guide surfaces (111D″) are inclined to direct tap water naturally toward the drainage holes for discharge. In an alternative configuration shown in FIG. 11, the outlet unit (111D) comprises a single drain pipe (111D’) with multiple drainage holes, each served by its own discharge guide surface (111D″).

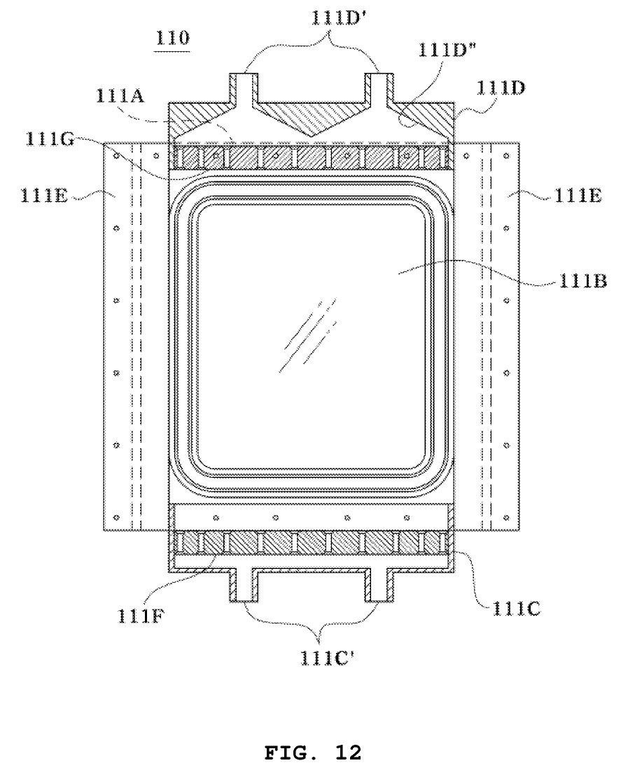

As shown in FIGS. 11 and 12, in the preferred embodiment, an outlet-side water distribution block (111G) is further installed horizontally inside the outlet unit (111D) or at the upper portion of the frames (111A). The block (111G) prevents tap water from flowing directly toward the drain pipes (111D’) and maintains a uniform flow rate until the water passes through its passage holes, compensating for the velocity increase that develops near the drain pipes as water rises through the flow cell. Together, the inlet-side block (111F) and outlet-side block (111G) ensure uniform flow distribution at both ends of the flow cell (110).

The cloud computing web server (20) receives detection data from the machine vision inspection device (10) in real time and organizes it into databases for statistical analysis and algorithm training. Algorithm updates are applied on a defined schedule or when new training data becomes available. The server (20) also provides detection data to the web platform (30), enabling remote real-time monitoring of multiple inspection devices (10) over an internet connection. When larval organisms are detected, event information is dispatched through the notification platform (40) via messaging services (e.g., KakaoTalk, Line, iMessage, or WhatsApp) or SMS, enabling users and administrators to receive and act on alerts regardless of location.

The analysis data visualization web platform (30) presents detection data as graphs and other visual formats on a webpage, organized by time, date, and location. Larva detection events are stored separately for rapid review by users or administrators.

The notification platform (40) is implemented as an application on a smartphone or tablet. When a larva detection alert arrives while the application is in the background, the device issues an audible alarm or vibration notification.

### ADVANTAGES OF THE INVENTION

Because the flow cell incorporates the inlet-side water distribution block internally, tap water is distributed uniformly throughout the flow cell without a separate external water distribution device, simplifying the overall system.

The oblong shape of the water passage holes resists blockage by foreign matter, and the progressively narrowing hole spacing from center to ends compensates for lower water pressure at positions distal from the inlet pipes, achieving a uniform flow rate across the full width of the flow cell. In the preferred embodiment, the additional outlet-side water distribution block further stabilizes flow uniformity at the outlet end, providing consistent imaging conditions for the vision camera(s).

The machine vision inspection device rapidly identifies target objects from the flowing tap water, and the AI object detection algorithm assigns a confidence score to each target object to classify it as a larva—identifying its species—or as non-larval foreign matter, enabling quick and reliable contamination monitoring.

1. A flow cell for real-time monitoring of microscopic larvae in tap water, the flow cell comprising:
a pair of main body frames, each formed in a rectangular plate shape having a through-hole in a center portion thereof, the pair of main body frames being installed spaced apart in a front-rear direction;
a pair of transparent glass plates covering the through-holes of the pair of main body frames, respectively;
a tap water inlet unit on one side of the main body frames configured to supply tap water between the pair of transparent glass plates; and
a tap water outlet unit on another side of the main body frames configured to discharge tap water from between the pair of transparent glass plates.

2. A flow cell for real-time monitoring of microscopic larvae in tap water, the flow cell comprising:
a pair of main body frames, each formed in a rectangular plate shape having a through-hole in a center portion thereof, the pair of main body frames being installed facing each other and spaced apart in a front-rear direction;
a pair of transparent glass plates covering the through-holes of the pair of main body frames;
a tap water inlet unit on one side of the main body frames configured to supply tap water to an interior of the flow cell;
a tap water outlet unit on another side of the main body frames configured to discharge tap water from the interior of the flow cell;
side sealing blocks installed along a height direction on both lateral sides between the pair of main body frames; and
an inlet-side water distribution block installed horizontally inside the tap water inlet unit or between the pair of main body frames, the inlet-side water distribution block having a plurality of water passage holes distributed along its length to distribute tap water across a width of the flow cell.

3. The flow cell of claim 2, wherein the tap water outlet unit comprises a discharge guide surface inclined to direct tap water toward drainage holes of a drain pipe.

4. The flow cell of claim 2, wherein the tap water outlet unit further comprises an outlet-side water distribution block installed horizontally and having a plurality of water passage holes to distribute discharged tap water across the width of the flow cell.

5. The flow cell of claim 2, wherein the water passage holes of the inlet-side water distribution block are spaced progressively more closely from a center of the block toward both ends thereof, such that the number of water passage holes per unit length is greater at the ends than at the center.

6. A machine vision system for real-time monitoring of microscopic larvae in tap water, the system comprising:
a machine vision inspection device having a flow cell installed therein, the machine vision inspection device being configured to continuously capture real-time images of tap water flowing through the flow cell using a vision camera, identify target objects in the captured images, and automatically classify each target object as a larva or non-larval foreign matter using an artificial intelligence object detection algorithm;
a cloud computing web server communicatively connected to the machine vision inspection device, the cloud computing web server being configured to receive larva detection data in real time, maintain a training database to improve performance of the artificial intelligence object detection algorithm, and transmit updated training data to the machine vision inspection device;
an analysis data visualization web platform configured to receive detection data from the machine vision inspection device through the cloud computing web server, convert the detection data organized by time, date, and location into graphical visualization data, and provide the graphical visualization data to users through a website; and
a notification platform configured to transmit real-time warning alerts regarding larva detection to registered users through the cloud computing web server.

7. The machine vision system of claim 6, wherein the machine vision inspection device comprises:
a housing having an internal accommodation space;
a flow cell installed inside the housing and connected to a water supply pipe so that tap water passes therethrough;
one or more vision cameras installed on one side of the flow cell to capture real-time images of tap water flowing through the flow cell;
a lighting device installed on a side of the flow cell opposite the one or more vision cameras to provide transmitted illumination for imaging;
an image preprocessing unit installed inside the housing to remove noise from captured images and isolate images of target objects;
an artificial intelligence object detection algorithm installed inside the housing to automatically classify each target object as a larva or non-larval foreign matter; and
a data transmission unit installed inside the housing and communicatively connected to a cloud computing web server to transmit larva detection data.

8. The machine vision system of claim 7, wherein the machine vision inspection device further comprises a display configured to output real-time images of tap water in the flow cell captured by the one or more vision cameras and images of larvae identified by the artificial intelligence object detection algorithm.

9. The machine vision system of claim 7, wherein the artificial intelligence object detection algorithm is configured to:
divide each preprocessed image into a grid of square areas;
detect a snap image of any area in which a target object is present;
assign a confidence score to the target object by comparing it against trained larva models; and
classify the target object as a larva when the confidence score meets or exceeds a classification threshold, and as non-larval foreign matter otherwise.

10. The machine vision system of claim 6, wherein the flow cell comprising:
a pair of main body frames, each formed in a rectangular plate shape having a through-hole in a center portion thereof, the pair of main body frames being installed facing each other and spaced apart in a front-rear direction;
a pair of transparent glass plates covering the through-holes of the pair of main body frames;
a tap water inlet unit on one side of the main body frames configured to supply tap water to an interior of the flow cell;
a tap water outlet unit on another side of the main body frames configured to discharge tap water from the interior of the flow cell;
side sealing blocks installed along a height direction on both lateral sides between the pair of main body frames; and
an inlet-side water distribution block installed horizontally inside the tap water inlet unit or between the pair of main body frames, the inlet-side water distribution block having a plurality of water passage holes distributed along its length to distribute tap water across a width of the flow cell.

11. The machine vision system of claim 10, wherein the water passage holes of the inlet-side water distribution block of the flow cell are spaced progressively more closely from a center of the block toward both ends thereof, such that the number of water passage holes per unit length is greater at the ends than at the center.

12. The machine vision system of claim 10, wherein the tap water outlet unit of the flow cell further comprises an outlet-side water distribution block installed horizontally and having a
plurality of water passage holes to distribute discharged tap water across the width of the flow cell.
